(19) United States
(12) Patent Application Publication
(10) Pub. No.: US 2026/0110653 A1
(43) Pub. Date: Apr. 23, 2026

(54) SOIL MOISTURE SENSOR ASSEMBLY FOR A SEED-PLANTING IMPLEMENT

(71) Applicant: CNH Industrial America LLC, New Holland, PA (US)

(72) Inventor: Michael Christopher Conboy, Chicago, IL (US)

(21) Appl. No.: 18/924,780

(22) Filed: Oct. 23, 2024

(51) Int. Cl. G01N 27/22; A01B 35/08; A01C 5/06; G01N 33/24

(52) U.S. Cl. CPC G01N 27/223; A01B 35/08; A01C 5/064; G01N 33/245; G01N 33/246

(57) ABSTRACT

A soil moisture sensor assembly for a seed-planting implement includes a furrow firmer configured to shape a furrow being formed in soil by the seed-planting implement, with the furrow firmer extending in a vertical direction from a top end to a bottom end. The furrow firmer defines a cavity at the bottom end. Furthermore, the assembly includes first and second electrodes positioned within the cavity for use in determining the soil moisture of the soil. Additionally, the assembly includes a non-electrically conductive housing positioned between the first and second electrodes and the furrow firmer in the vertical direction.

### FIELD OF THE INVENTION

The present disclosure generally relates to seed-planting implements and, more particularly, to a soil moisture sensor assembly for a seed-planting implement.

### BACKGROUND OF THE INVENTION

Modern farming practices strive to increase yields of agricultural fields. In this respect, seed-planting implements are towed behind a tractor or other work vehicle to disperse seed throughout a field. For example, a seed-planting implement typically includes one or more furrow-forming tools, such as one or more disk openers, that excavate a furrow or trench in the soil. One or more dispensing devices of the seed-planting implement may, in turn, deposit the seeds into the furrow(s). After deposition of the seeds, a furrow-closing assembly may close the furrow in the soil, such as by pushing the excavated soil into the furrow.

The soil moisture of the soil within the field is an important parameter when controlling the operation of the seed-planting implement. In this respect, soil moisture sensors and sensor assemblies for seed-planting implements have been developed. While such sensors and sensor assemblies work well, further improvements are needed.

Accordingly, an improved soil moisture sensor assembly for a seed-planting implement would be welcomed in the technology.

### SUMMARY OF THE INVENTION

Aspects and advantages of the technology will be set forth in part in the following description or may be obvious from the description, or may be learned through practice of the technology.

In one aspect, the present subject matter is directed to a soil moisture sensor assembly for a seed-planting implement. The assembly includes a furrow firmer configured to shape a furrow formed in soil by the seed-planting implement, with the furrow firmer extending in a vertical direction from a top end to a bottom end. The furrow firmer defines a cavity at the bottom end. Furthermore, the assembly includes first and second electrodes positioned within the cavity for use in determining the soil moisture of the soil. Additionally, the assembly includes a non-electrically conductive housing positioned between the first and second electrodes and the furrow firmer in the vertical direction.

In another aspect, the present subject matter is directed to a row unit for a seed-planting implement. The row unit includes a row unit frame and a disk opener rotatably coupled to the row unit frame, with the disk opener configured to form a furrow within soil of a field as the seed-planting implement travels across the field. Moreover, the row unit includes a furrow firmer coupled to the row unit frame, with the furrow firmer configured to shape the furrow, with the furrow firmer extending in a vertical direction from a top end to a bottom end. The furrow firmer defines a cavity at the bottom end. In addition, the row unit includes first and second electrodes positioned within the cavity for use in determining the soil moisture of the soil. Furthermore, the assembly includes a non-electrically conductive housing positioned between the first and second electrodes and the furrow firmer in the vertical direction.

In a further aspect, the present subject matter is directed to a seed-planting implement including a toolbar and a plurality of row units supported on the toolbar. At least one row unit of the plurality of row units includes a furrow firmer configured to shape a furrow formed in soil by the seed-planting implement, with the furrow firmer extending in a vertical direction from a top end to a bottom end. Furthermore, the furrow firmer defines a cavity at the bottom end. Additionally, the at least one row unit of the plurality of row units includes first and second electrodes positioned within the cavity for use in determining the soil moisture of the soil. Moreover, the at least one row unit of the plurality of row units includes a non-electrically conductive housing positioned between the first and second electrodes and the furrow firmer in the vertical direction.

These and other features, aspects, and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which refers to the appended figures, in which:

FIG. 1 illustrates a perspective view of one embodiment of a seed-planting implement in accordance with aspects of the present subject matter;

FIG. 2 illustrates a side view of one embodiment of a row unit of a seed-planting implement in accordance with aspects of the present subject matter;

FIG. 3 illustrates a bottom view of one embodiment of a soil moisture sensor assembly in accordance with aspects of the present subject matter;

FIG. 4 illustrates a cross-sectional view of the soil moisture sensor assembly taken generally about Line 4-4 in FIG. 3; and

FIG. 5 illustrates a cross-sectional view of the soil moisture sensor assembly taken generally about Line 5-5 in FIG. 3.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

As used herein, the term “and/or,” when used in a list of two or more items, means that any one of the listed items can be employed by itself, or any combination of two or more of the listed items can be employed. For example, if a composition or assembly is described as containing components A, B, and/or C, the composition or assembly can contain A alone; B alone; C alone; A and B in combination; A and C in combination; B and C in combination; or A, B, and C in combination.

In general, the present subject matter is directed to a soil moisture sensor assembly for a seed-planting implement. As will be described below, the seed-planting implement includes a furrow firmer configured to shape the furrow formed in the soil by the row unit. In this respect, the furrow firmer extends in a vertical direction from a top end to a bottom end such that the furrow firmer defines a cavity at the bottom end.

Additionally, the soil moisture sensor assembly includes first and second electrodes and a non-electrically conductive housing. Specifically, in several embodiments, the first and second electrodes are positioned within the cavity of the furrow firmer for use in determining the soil moisture of the soil. Moreover, the non-electrically conductive housing is positioned between the first and second electrodes and the furrow firmer in the vertical direction. In this respect, the non-electrically conductive housing electrically isolates the first and second electrodes from the furrow firmer. For example, the first and second electrodes may be a metallic material, such as first and second metallic strips, and the non-electrically conductive housing may be formed of a polymeric material.

The disclosed soil moisture sensor improves the operation of the seed-planting implement. More specifically, as described above, the disclosed soil moisture sensor includes a non-electrically conductive housing electrically, which isolates the first and second electrodes from the furrow firmer. In this respect, the disclosed soil moisture sensor assembly is positioned within a furrow firmer of the seed-planting implement. Thus, the disclosed soil moisture sensor assembly does not disturb furrow closing operation unlike conventional soil moisture sensors that bolt onto the seed-planting implement behind the furrow firmers. This, in turn, improves the agricultural performance of the field.

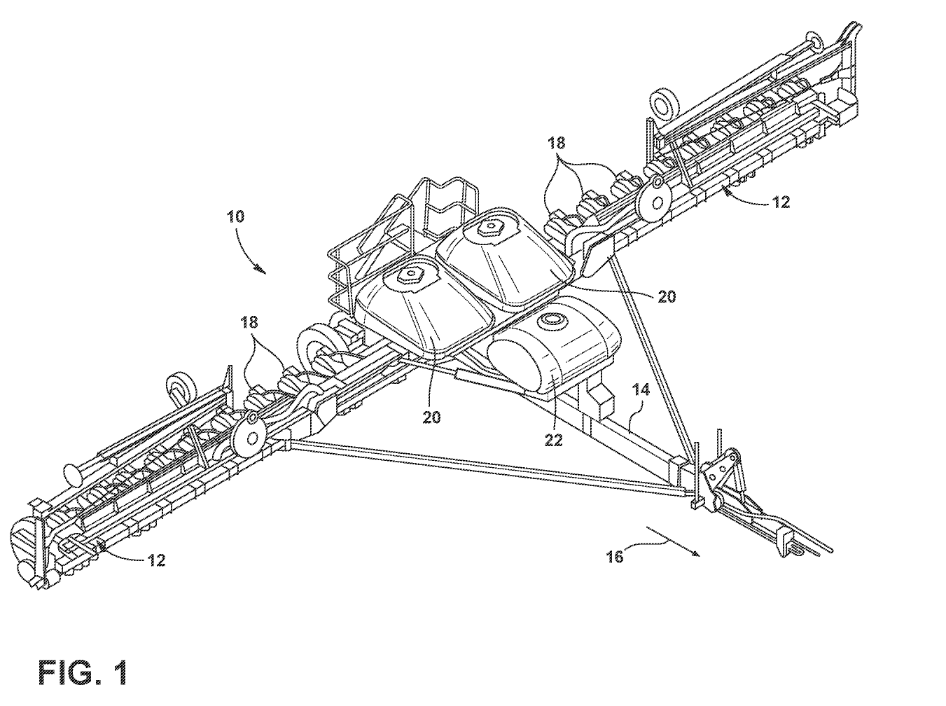

Referring now to drawings, FIG. 1 illustrates a perspective view of one embodiment of a seed-planting implement 10. In the illustrated embodiment, the seed-planting implement 10 is configured as a planter. However, in alternative embodiments, the seed-planting implement 10 may be configured as a seeder, a strip-tiller, a side-dresser, or any other suitable agricultural implement that deposits seeds into a field.

As shown in FIG. 1, the seed-planting implement 10 may include a laterally extending toolbar 12. More specifically, the toolbar 12 is connected at its middle to a forwardly extending tow bar 14 to allow the seed-planting implement 10 to be towed by a work vehicle (not shown), such as an agricultural tractor, in a direction of travel 16. In this respect, the toolbar 12 is generally configured to support a plurality of seed planting units or row units 18. Each row unit 18, in turn, is configured to deposit seeds at a desired depth beneath the soil surface and with a desired seed spacing as the seed-planting implement 10 travels across the field in the direction of travel 16, thereby establishing rows of planted seeds. In some embodiments, the bulk of the seeds to be planted may be stored in one or more hoppers or seed tanks 20. Thus, as seeds are planted by the row units 18, a pneumatic distribution system may distribute additional seeds from the seed tanks 20 to individual row units 18. Additionally, one or more fluid tanks 22 may store agricultural fluids, such as insecticides, herbicides, fungicides, fertilizers, and/or the like. These fluids, in turn, may be supplied to the row units 18 for spraying onto the seeds during planting.

For purposes of illustration, only a portion of the row units 18 of the seed-planting implement 10 has been shown in FIG. 1. In general, the seed-planting implement 10 may include any number of row units 18, such as 6, 8, 12, 16, 24, 32, or 36 row units. In addition, the lateral spacing between row units 18 may be selected based on the type of crop being planted. For example, the row units 18 may be spaced approximately 30 inches from one another for planting corn, and approximately 15 inches from one another for planting soybeans.

The configuration of the seed-planting implement 10 described above and shown in FIG. 1 is provided only to place the present subject matter in an exemplary field of use. Thus, the present subject matter may be readily adaptable to any manner of seed-planting implement configuration.

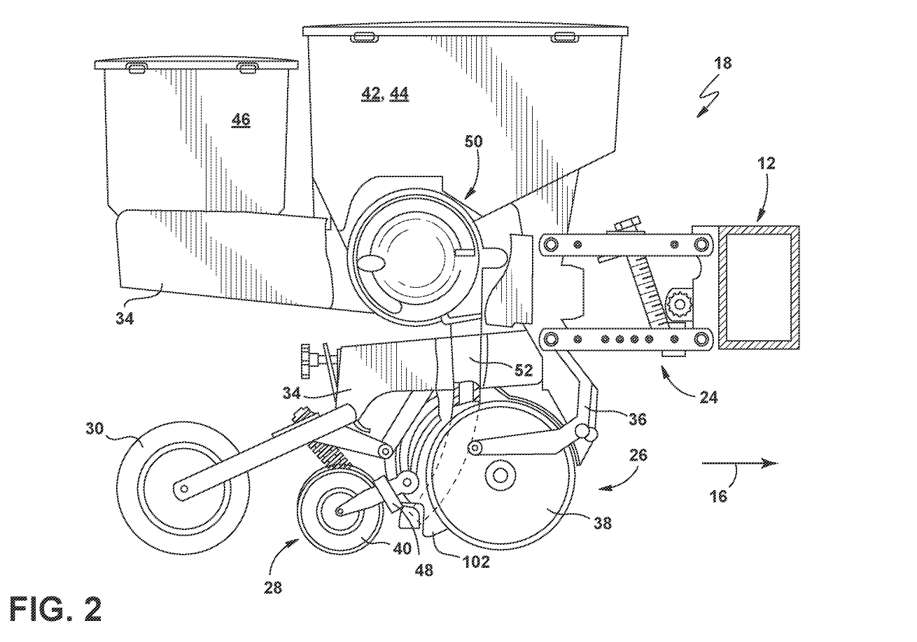

FIG. 2 illustrates a side view of one embodiment of a row unit 18. As shown, the row unit 18 includes a linkage assembly 24 configured to mount the row unit 18 to the toolbar 12 of the seed-planting implement 10. Furthermore, the row unit 18 also includes a row unit frame 34. In this respect, the row unit 18 may include a furrow opening assembly 26, a furrow closing assembly 28, and a press wheel 30 supported on or otherwise coupled row unit frame 34. In general, the furrow opening assembly 26 may include a gauge wheel (not shown) operatively coupled to the row unit frame 34 via a support arm 36. Additionally, the opening assembly 26 may also include one or more disk openers 38 rotatably coupled to the row unit frame 34. Moreover, the row unit 18 includes a furrow firmer 102 coupled to the row unit frame 34. The gauge wheel is not shown in FIG. 2 to better illustrate the disk opener(s) 38 and furrow firmer 102. The disk opener(s) 38 is configured to form or otherwise excavate a furrow or trench within the soil of a field as the seed-planting implement 10 (FIG. 1) travels across the field in the direction of travel 16. In this respect, the furrow firmer 102 is configured to shape the furrow formed in soil by the disk opener(s) 38 and firm the walls of such firm to prevent premature collapse of the furrow. In addition, the gauge wheel is configured to roll along or otherwise engage the surface of the field such that the position of the gauge wheel relative to the row unit frame 34 sets the depth of the furrow being excavated. Furthermore, as shown, the furrow closing assembly 28 may include a closing disk(s) 40 configured to close or collapse the furrow after seeds have been deposited therein. Thereafter, the press wheel 30 may roll over the closed furrow to firm the soil over the seed and promote favorable seed-to-soil contact.

Additionally, as shown in FIG. 2, the row unit 18 may include one or more seed hoppers 42, 44 and a fluid tank 46 supported on the row unit frame 34. In general, the seed hopper(s) 42, 44 may be configured to store seeds received from the seed tanks 20, which are to be deposited within the furrow as the row unit 18 travels across the field. For instance, in several embodiments, the row unit 18 may include a first seed hopper 42 configured to store seeds of a first seed type and a second hopper 44 configured to store seeds of a second seed type. However, both seed hoppers 42, 44 may be configured to store the same type of seeds. Furthermore, the fluid tank 46 may be configured to store fluid received from the fluid tank 22 (FIG. 1), which is to be sprayed onto the seeds dispensed from the seed hoppers 42, 44. For example, a sprayer assembly 48 mounted on the row unit frame 34 may be configured to spray the fluid stored in the fluid tank 22 onto the seeds.

Moreover, the row unit 18 may include a seed meter 50 supported on the row unit frame 34. In general, the seed meter 50 is configured to uniformly release seeds received from the seed hopper(s) 42, 44 for deposition within the furrow. For instance, in one embodiment, the seed meter 50 may be coupled to a suitable vacuum source (e.g., a blower powered by a motor and associated tubing or hoses) configured to generate a vacuum or negative pressure that attaches the seeds to a rotating seed disk of the seed meter 50, which controls the rate at which the seeds are output from the seed meter 50 to an associated seed tube 52. As shown in FIG. 2, the seed tube 52 may extend vertically from the seed meter 50 toward the ground to facilitate delivery of the seeds discharged from the seed meter 50 to the furrow.

The configuration of the row unit 18 described above and shown in FIG. 2 is provided only to place the present subject matter in an exemplary field of use. Thus, the present subject matter may be readily adaptable to any manner of seed planting unit configuration.

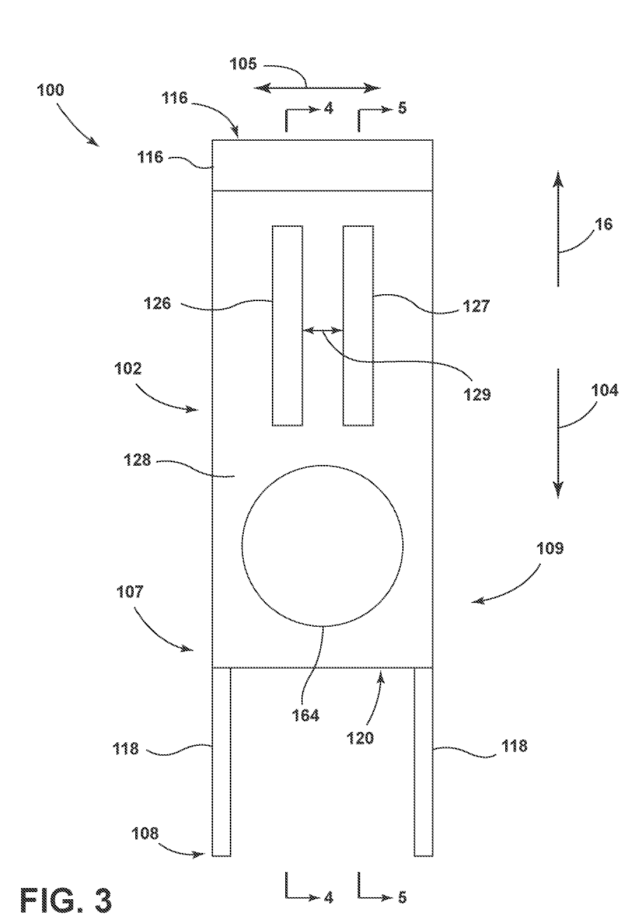

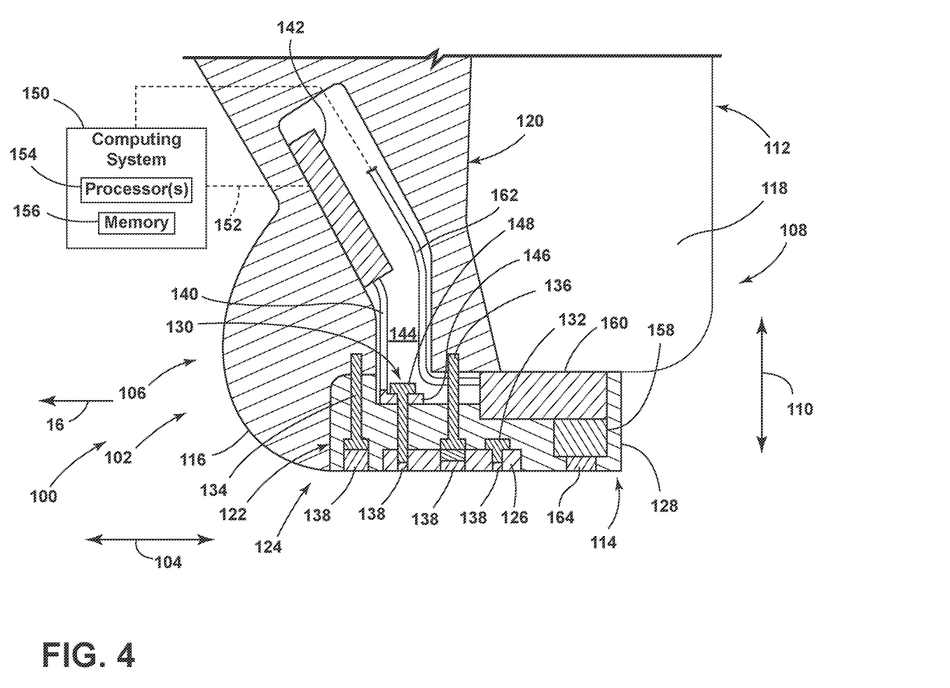

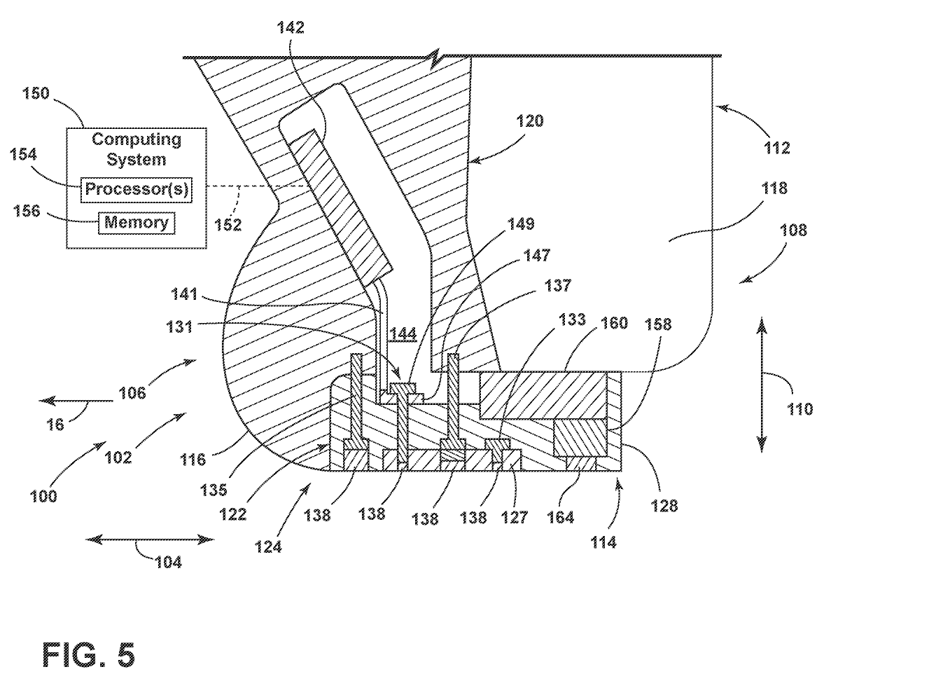

FIGS. 3-5 illustrate differing views of one embodiment of a soil moisture sensor assembly100 for a seed-planting implement. Specifically, FIG. 3 illustrates a bottom view of the soil moisture sensor assembly 100. Furthermore, FIG. 4 illustrates a cross-sectional view of the soil moisture sensor assembly 100 taken generally about Line 4-4 in FIG. 3. Moreover, FIG. 5 illustrates a cross-sectional view of the soil moisture sensor assembly 100 taken generally about Line 5-5 in FIG. 3.

In general, the soil moisture sensor assembly 100 will be described herein with reference to the seed-planting implement 10 and the row unit 18 described above with reference to FIGS. 1 and 2. However, the disclosed soil moisture sensor assembly 100 can generally be utilized with seed-planting implements having any other suitable implement configuration and/or row units having any other suitable row unit configuration.

As shown in FIGS. 3-5, the soil moisture sensor assembly 100 includes the furrow firmer 102 of the row unit 18. More specifically, the furrow firmer 102 extends in a longitudinal direction 104 from a forward end 106 to an aft end 108, with the longitudinal direction 104 extending generally parallel to the direction of travel 16. Furthermore, as shown in FIG. 3, the furrow firmer 102 extends in a lateral direction 105 from a first side 107 to a second side end 109, with the lateral direction 105 extending generally perpendicular to the longitudinal direction 104. Additionally, as shown in FIGS. 4 and 5, the furrow firmer 102 extends in a vertical direction 110 from a top end 112 to a bottom end 114, with the vertical direction 110 extending generally perpendicular to the longitudinal direction 104 and the lateral direction 105. In some embodiments, the furrow firmer 102 includes a body 116 (e.g., a metallic casting) and a sleeve 118 (e.g., formed of sheet metal) coupled to an aft end 120 of the body 116. Additionally, the furrow firmer 102 defines a cavity 122 at the bottom end 114. For example, as shown in FIGS. 4 and 5, in the illustrated embodiment, the cavity 122 is defined at a bottom end 124 of the body 116. As will be described below, additional components of the soil moisture sensor assembly 100 are positioned within the cavity 122, thereby allowing for the determination of the soil moisture of the soil while not negatively impacting the furrow closing operation being performed by the closing disk(s) 40 (FIG. 2).

Referring to FIGS. 3-5, the soil moisture sensor assembly 100 includes first and second electrodes 126, 127 positioned within the cavity 122. More specifically, the first and second electrodes 126, 127 extend in the longitudinal direction 104 between the forward and aft ends 106, 108 of the furrow firmer 102. Moreover, the first and second electrodes 126, 127 are spaced apart from each other in the lateral direction 105. As such, a gap 129 is defined between the first electrode 126 and the second electrode 127 in the lateral direction 105. Additionally, the first and second electrodes 126, 127 are positioned such that the first and second electrodes 126, 127 contact the soil forming the bottom surface of the furrow as the row unit 18 travels across the field in the direction of travel 16. In this respect, and as will be described below, the first and second electrodes 126, 127 are used in determining the soil moisture of the soil forming the furrow. Thus, in several embodiments, the first and second electrodes 126, 127 are formed of a metallic material. For example, in some embodiments, the first and second electrodes 126, 127 are configured as first and second metallic strips or plates.

Additionally, the soil moisture sensor assembly 100 includes a non-electrically conductive housing 128. As shown, the non-electrically conductive housing 128 is positioned within the cavity 122. Moreover, the non-electrically conductive housing 128 is positioned between the first and second electrodes 126, 127 and at least a portion of the furrow firmer 102 in the vertical direction 110. In this respect, the non-electrically conductive housing 128 electrically isolates the first and second electrodes 126, 127 from the furrow firmer 102. Thus, the non-electrically conductive housing 128 allows the first and second electrodes 126, 127 to be positioned within the furrow firmer 102 without shorting on the body 116 or the sleeve 118, thereby improving the furrow closing operation.

The non-electrically conductive housing 128 may be formed out of any suitable non-electrically conductive or otherwise electrically insulative material. For example, in some embodiments, the non-electrically conductive housing 128 may be formed of a polymeric material.

Moreover, the first and second electrodes 126, 127 and the non-electrically conductive housing 128 may be mechanically coupled to the furrow firmer 102 in any suitable manner. More specifically, in some embodiments, the first electrode 126 may be mechanically coupled to the non-electrically conductive housing 128 via one or more fasteners. For example, as shown in FIGS. 4, in the illustrated embodiment, the first electrode 126 is mechanically coupled to the non-electrically conductive housing 128 via a first fastener 130 and a second fastener 132. Similarly, in some embodiments, the second electrode 127 may be mechanically coupled to the non-electrically conductive housing 128 via one or more fasteners. For example, as shown in FIG. 5, in the illustrated embodiment, the second electrode 127 is mechanically coupled to the non-electrically conductive housing 128 via a third fastener 131 and a fourth fastener 133. Moreover, the non-electrically conductive housing 128 may be mechanically coupled to the furrow firmer 102 via one or more fasteners. For example, as shown in FIGS. 4 and 5, in the illustrated embodiment, the non-electrically conductive housing 128 is mechanically coupled to the furrow firmer 102 (e.g., the body 116 of the furrow firmer 102) via a fifth fastener 134 (FIG. 4), a sixth fastener 136 (FIG. 4), a seventh fastener 135 (FIG. 5), and an eighth fastener 137 (FIG. 5). In some embodiments, caps 138 may be placed in the holes in which the first, second, third, fourth, fifth, sixth, seventh, and/or eighth fasteners 130, 132, 134, 136, 135, 137 are received to prevent soil accumulation therein.

In addition, the soil moisture sensor assembly 100 includes first and second wires 140, 141 and a circuit board 142. More specifically, the furrow firmer 102 and/or the non-electrically conductive housing 128 may define a passage 144. In this respect, the circuit board 142 may be positioned within an upper portion of the passage 144, and the first and second wires 140, 141 may be routed at least partially through the passage 144. For example, as shown in FIG. 4, one end of the first wire 140 may be electrically coupled to the circuit board 142, while a first terminal 146 positioned within the cavity 122 may be mechanically coupled to the opposing end of the first wire 140. Similarly, as shown in FIG. 5, one end of the second wire 141 may be electrically coupled to the circuit board 142, while a second terminal 147 positioned within the cavity 122 may be mechanically coupled to the opposing end of the second wire 141. In this respect, one of the fasteners coupling the first electrode 126 and the non-electrically conductive housing 128 may electrically couple the first terminal 146 and the first electrode 126. For example, as shown in FIG. 4, in the illustrated embodiment, at least a portion of the first terminal 146 is positioned between a head 148 of the first fastener 130 and the non-electrically conductive housing 128 in the vertical direction 110. Thus, electric current may flow from the first electrode 126 through the first fastener 130 and into the first terminal 146 before flowing through the first wire 140 to the circuit board 142. Alternatively, electric current may flow in the opposite direction. Similarly, one of the fasteners coupling the second electrode 127 and the non-electrically conductive housing 128 may electrically couple the second terminal 147 and the second electrode 127. For example, as shown in FIG. 5, at least a portion of the second terminal 147 is positioned between a head 149 of the third fastener 131 and the non-electrically conductive housing 128 in the vertical direction 110. Thus, electric current may flow from the second electrode 127 through the third fastener 131 and into the second terminal 147 before flowing through the second wire 141 to the circuit board 142. Alternatively, electric current may flow in the opposite direction.

Moreover, the soil moisture sensor assembly 100 includes a computing system 150 communicatively coupled to one or more components of the soil moisture sensor assembly 100, the row unit 18, and/or the seed-planting implement 10. For instance, in some embodiments, the computing system 150 may be communicatively coupled to the circuit board 142 via a communicative link 152. Alternatively, the circuit board 142 may be part of the computing system 150. As such, the computing system 150 may be configured to receive electric current or other data from the first and/or second electrodes 126, 127 (or the circuit board 142) via the first and/or second wires 140, 141 and/or the communicative link 152. Such electric current or data may generally be indicative of the soil moisture of the soil within the field. In addition, the computing system 150 may be communicatively coupled to any other suitable components of the soil moisture sensor assembly 100, the row unit 18, and/or the seed-planting implement 10, such as any other sensor(s) positioned within the cavity 122.

In general, the computing system 150 may include one or more processor-based devices, such as a given controller or computing device or any suitable combination of controllers or computing devices. Thus, in several embodiments, the computing system 150 may include one or more processor(s) 154 and associated memory device(s) 156 configured to perform a variety of computer-implemented functions. As used herein, the term “processor” refers not only to integrated circuits referred to in the art as being included in a computer, but also refers to a controller, a microcontroller, a microcomputer, a programmable logic circuit (PLC), an application specific integrated circuit, and other programmable circuits. Additionally, the memory device(s) 156 of the computing system 150 may generally comprise memory element(s) including, but not limited to, a computer-readable medium (e.g., random access memory RAM)), a computer-readable non-volatile medium (e.g., a flash memory), a floppy disk, a compact disk-read only memory (CD-ROM), a magneto-temperature disk (MOD), a digital versatile disk (DVD) and/or other suitable memory elements. Such memory device(s) 156 may generally be configured to store suitable computer-readable instructions that, when implemented by the processor(s) 154, configure the computing system 150 to perform various computer-implemented functions. In addition, the computing system 150 may also include various other suitable components, such as a communications circuit or module, one or more input/output channels, a data/control bus, and/or the like.

The various functions of the computing system 150 may be performed by a single processor-based device or may be distributed across any number of processor-based devices, in which instance such devices may be considered to form part of the computing system 150. For instance, the functions of the computing system 150 may be distributed across multiple application-specific controllers or computing devices (e.g., the circuit board 142 may be part of the computing system 150).

In several embodiments, the computing system 150 may use the first and second electrodes 126, 127 to determine the soil moisture content of the field. More specifically, one of the first or second electrodes 126, 127 may act as a positive capacitor plate, while the other of the first or second electrodes 126, 127 may act as a negative capacitor plate. In this respect, the soil (and the moisture therein) acts as a dielectric between the first and second electrodes 1126, 127. As such, the first and second electrode 126, 127 and the soil effectively form a capacitor having a capacitance that varies based on the moisture content of the soil. Furthermore, as mentioned above, the computing system 150 is electrically coupled to the first and second electrodes 126, 127. In this respect, the computing system 150 may supply electric current (e.g., in the form of a square sine wave) to one of the first or second electrodes 126, 127 via the corresponding first or second wire 140, 141. Based on a voltage present across a circuit (e.g., a 555-based circuit) electrically the first and second electrodes 126, 127, the soil moisture content of the soil can be determined. For example, the computing system 150 includes a look-up stored within its memory device(s) 156 correlating the voltage produced in the circuit coupled to the first and second electrodes 126, 127 with a soil moisture content value for the soil. The circuit may be part of the computing system 150 or separate from the computing system 150.

Furthermore, as indicated above, other sensors may be positioned within the cavity 122 defined by the furrow firmer. For example, as shown in FIGS. 4 and 5, in some embodiments, a temperature sensor 158 may be positioned within the cavity 122. In general, the temperature sensor 158 may be configured to generate data indicative of the temperature of the soil. More specifically, the temperature sensor 158 (e.g., a thermopile) may be coupled to the non-electrically conductive housing 128. For example, in one embodiment, the temperature sensor 158 may be coupled to a circuit board 160 that, in turn, is coupled (e.g., potted) to the non-electrically conductive housing 128. A wire 162 may be coupled (e.g., soldered) to the circuit board 160 and routed through the passage 144 for eventual direct or indirect coupling to the computing system 150. In alternative embodiments, the circuit board 160 may be considered part of the computing system 150 and the wire 162 may be part of the communicative link 152. Additionally, a lens 164 may be positioned within the cavity 122 and coupled (e.g., adhesively coupled) to the non-electrically conductive housing 128. As such, the temperature sensor 158 has a field of view through the lens 164 that is directed at the bottom surface of the furrow. That is, the temperature sensor 158 can view the soil defining the bottom surface of the furrow through the lens 164. In this respect, the temperature sensor 158 is at least partially positioned between the circuit board 160 and the lens 164 in the vertical direction 110. However, in alternative embodiments, the temperature sensor 158 may be omitted, and/or other types of sensors may be positioned within the cavity 122.

This written description uses examples to disclose the technology, including the best mode, and also to enable any person skilled in the art to practice the technology, including making and using any devices or systems and performing any incorporated methods. The patentable scope of the technology is defined by the claims and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. A soil moisture sensor assembly for a seed-planting implement, the soil moisture sensor assembly comprising:
a furrow firmer configured to shape a furrow being formed in soil by the seed-planting implement, the furrow firmer extending in a vertical direction from a top end to a bottom end, the furrow firmer defining a cavity at the bottom end;
first and second electrodes positioned within the cavity for use in determining a soil moisture of the soil; and
a non-electrically conductive housing positioned between the first and second electrodes and the furrow firmer in the vertical direction.

2. The soil moisture sensor assembly of claim 1, wherein the non-electrically conductive housing electrically isolates the first and second electrodes from the furrow firmer.

3. The soil moisture sensor assembly of claim 1, wherein at least one of the furrow firmer or the non-electrically conductive housing further defines a passage, the assembly further comprising:
first and second wires extending through the passage and electrically coupled to the first and second electrodes, respectively.

4. The soil moisture sensor assembly of claim 3, further comprising:
first and second terminals positioned within the cavity and mechanically coupled to ends of the first and second wires, respectively.

5. The soil moisture sensor assembly of claim 4, further comprising:
first and second fasteners mechanically coupling the first and second electrodes to the non-electrically conductive housing, respectively, the first and second fasteners further electrically coupling the first and second terminals to the first and second electrodes, respectively.

6. The soil moisture sensor assembly of claim 5, wherein:
at least a portion of the first terminal is positioned between a head of the first fastener and the non-electrically conductive housing in the vertical direction; and
at least a portion of the second terminal is positioned between a head of the second fastener and the non-electrically conductive housing in the vertical direction.

7. The soil moisture sensor assembly of claim 1, wherein the non-electrically conductive housing is mechanically coupled to the furrow firmer via one or more fasteners.

8. The soil moisture sensor assembly of claim 1, wherein the first and second electrodes are mechanically coupled to the non-electrically conductive housing via a plurality of fasteners.

9. The soil moisture sensor assembly of claim 1, wherein the non-electrically conductive housing is formed of a polymeric material and the first and second electrodes are formed of a metallic material.

10. The soil moisture sensor assembly of claim 1, wherein the first and second electrodes are first and second metallic strips.

11. A row unit for a seed-planting implement, the row unit comprising:
a row unit frame;
a disk opener rotatably coupled to the row unit frame, the disk opener configured to form a furrow within soil of a field as the seed-planting implement travels across the field;
a furrow firmer coupled to the row unit frame, the furrow firmer configured to shape the furrow, the furrow firmer extending in a vertical direction from a top end to a bottom end, the furrow firmer defining a cavity at the bottom end;
first and second electrodes positioned within the cavity for use in determining a soil moisture of the soil; and
a non-electrically conductive housing positioned between the first and second electrodes and the furrow firmer in the vertical direction.

12. The row unit of claim 11, wherein the non-electrically conductive housing electrically isolates the first and second electrodes from the furrow firmer.

13. The row unit of claim 11, wherein at least one of the furrow firmer or the non-electrically conductive housing further defines a passage, the assembly further comprising:
first and second wires extending through the passage and electrically coupled to the first and second electrodes, respectively.

14. The row unit of claim 13, further comprising:
first and second terminals positioned within the cavity and mechanically coupled to ends of the first and second wires, respectively.

15. The row unit of claim 14, further comprising:
first and second fasteners mechanically coupling the first and second electrodes to the non-electrically conductive housing, respectively, the first and second fasteners further electrically coupling the first and second terminals to the first and second electrodes, respectively.

16. The row unit of claim 15, wherein:
at least a portion of the first terminal is positioned between a head of the first fastener and the non-electrically conductive housing in the vertical direction; and
at least a portion of the second terminal is positioned between a head of the second fastener and the non-electrically conductive housing in the vertical direction.

17. The row unit of claim 11, wherein the non-electrically conductive housing is mechanically coupled to the furrow firmer via one or more fasteners.

18. The row unit of claim 11, wherein the first and second electrodes are mechanically coupled to the non-electrically conductive housing via a plurality of fasteners.

19. The row unit of claim 11, further comprising a temperature sensor positioned within the cavity.

20. A seed-planting implement, comprising:
a toolbar; and
a plurality of row units supported on the toolbar, at least one row unit of the plurality of row units comprising:
a furrow firmer configured to shape a furrow being formed in soil by the seed-planting implement, the furrow firmer extending in a vertical direction from a top end to a bottom end, the furrow firmer defining a cavity at the bottom end;
first and second electrodes positioned within the cavity for use in determining a soil moisture of the soil; and
a non-electrically conductive housing positioned between the first and second electrodes and the furrow firmer in the vertical direction.
